(19) United States
(12) Patent Application Publication
(10) Pub. No.: US 2004/0014678 A1
(43) Pub. Date: Jan. 22, 2004

(54) Prevention of beta-amyloid neurotoxicity by blockade of the ubiquitin-proteasome proteolytoc pathway

(76) Inventors: Antonella Favit, Lawrenceville, NJ (US); Maurizio Grimaldi, Bethesda, MD (US); Daniel L. Alkon, Bethesda, MD (US)

Correspondence Address: Venable, PO Box 34385, Washington, DC, 20043-9998, US

(21) Appl. No.: 10/344,258

(22) PCT Filed: Aug. 8, 2001
(86) PCT No.: PCT/US01/41619

(51) Int. Cl. A61K 38/04

(52) U.S. Cl. 514/19

(57) ABSTRACT

Compositions and methods for inhibiting neurotoxic ubiquitin-proteasome proteolysis, and treatment of neurodegenerative disorders. In particular, a method of suppressing the neurotoxic effect of β-amyloid peptide, by administering an inhibitor to block β-amyloid peptide-mediated ubiquitin-proteasome proteolysis. Preferred inhibitors are inhibitors of ubiquitination, such as leucine-alanine dipeptide, and inhibitors of post-ubiquitination proteasome activity, such as lactacystin.

### BACKGROUND OF THE INVENTION

1. Field of the Invention

The invention relates to compositions and methods for treating neurological disorders. In particular, the invention relates to a method of suppressing the neurotoxic effect of β-amyloid peptide, comprising administering an inhibitor to block β-amyloid peptide-mediated ubiquitin-proteasome proteolysis.

2. Background Information

Amyloid precursor protein (APP) in its natural condition may help stimulate neuronal growth factors. However, when amyloid precursor protein is cleaved prematurely, amyloid precursor protein fragments are formed, including β-amyloid peptide (βAP), which has been reported to cause apoptosis, reactive oxygen species-mediated cell damage, and necrosis of neurons. In Alzheimer's disease (AD), β-amyloid peptide aggregates and forms senile or β-amyloid plaques, a hallmark of the disease. β-amyloid peptide is believed to be responsible for neuronal death, but the precise mechanism is not known.

Prior references relate to the pre-β-amyloid peptide formation phase, whereby amyloid precursor protein is cleaved to β-amyloid peptide. It is known that proteasome/ubiquitin mediated proteolysis is involved in conversion of amyloid precursor protein to produce β-amyloid peptide. Administration of inhibitors has been shown to inhibit proteolysis of amyloid precursor protein, thereby preventing cleavage into fragments such as β-amyloid peptide. Lactacystin analogs may be used to selectively inhibit proteasome activity and treat Alzheimer's disease, by reducing formation of β-amyloid peptide. (U.S. Pat. No. 5,756,764 issued to Fenteany, col. 57, line 62; Fenteany et al. (1995). A reverse effect has also been noted, where β-amyloid peptide secretion was inhibited, and amyloid precursor protein buildup was seen (Marambaud et al., 1997). Honda et al. (1999) observed that inhibition of proteasomes increased the secretion of β-amyloid peptide and suggested that the ubiquitin-proteasome pathway is dysfunctional in Alzheimer's disease. Yamazaki et al. (1997) have also shown that proteasomes control the level of secretion of β-amyloid peptide.

Deterioration of intracellular proteins due to aging or damage by various insults may be signaled by the covalent attachment of one or more ubiquitin molecules. (Laney et al. (1999)). Ubiquitination involves at least three tightly regulated enzymes that act in succession. (Ciechanover et al. (1994)). As a result of their ubiquitination, proteins are targeted for proteolysis by the proteasome, a multicomponent, energy-dependent, high-molecular-weight intracellular proteolytic organelle. (Ciechanover (1994)).

In the central nervous system (CNS), proteasome-mediated protein degradation plays a major role in the breakdown of cellular proteins damaged by oxidative stress or other insults causing glucose and oxygen shortage. (Alves-Rodrigues et al. (1998)). In many neurodegenerative disorders, inclusions containing ubiquitinated proteins are commonly found due to either defective activity of the proteasome or decreased energy levels that impair proteasome activity. (Lenmox et al. (1988); Lowe et al. (1988); Alves-Rodrigues et al., 1998). Furthermore, during conditions of stress, when general intracellular protein degradation in the CNS increases, the proteasome complex becomes particularly active. (Alves-Rodriguez et al., 1998).

Amyloid precursor protein (APP) and its related catabolic products have been implicated in the pathogenesis of Alzheimer's disease. (Wilson et al. (1999)). APP fragments, including β-amyloid, have been reported to cause apoptosis, reactive oxygen species-mediated cell damage, and necrosis of neurons. (Davis (1996); Suzuki (1997); Mattson et al. (1998); Yan et al. (1999)). β-amyloid peptide mediates PKCα and PKCγ degradation, which may be blocked by the selective proteasome inhibitor lactacystin in human fibroblasts. (Favit et al., 1998). However, the mechanism through which exposure to β-amyloid causes this broad spectrum of toxic activity has not been elucidated. (Vickers et al. (2000)).

The production of APP catabolic products has been suggested as a critical factor in the cascade of events leading to neurodegeneration. (Martin, 1999; Vickers et al., 2000). To this group of APP toxic derivatives belong several peptides that have been designated β-amyloid peptides. Two principal forms of β-amyloid peptides, corresponding to the region 1-40 and 1-42 of the APP molecule, are produced and accumulated in Alzheimer's brain. (Martin, 1999). These two β-amyloid peptides exert toxic activity on cultures of brain neurons derived from both the hippocampus and cortex. The mechanisms by which these peptides cause neuronal degeneration and death have been extensively studied. (Suzuki, 1997; Mattson and Pedersen, 1998: Yan et al., 1999). However, a critical mechanism triggered by β-amyloid, the blockage of which can ultimately prevent or reduce β-amyloid toxicity, has not been identified.

Enhanced oxidation and reduced energy levels associated with increased proteasome activity are commonly induced by β-amyloid in vitro. (Davis, 1996; Suzuki, 1997; Yan et al., 1999). Furthermore, proteasome activity is altered in many neurological disorders in which an excess of APP-related metabolites is associated with ubiquitin immunoreactivity in the typical lesions. (Lennox et al., 1988; Lowe et al., 1988; Alves-Rodrigues et al., 1998).

Recently, an association between a dysfunction of proteasome activity and neurodegeneration has been hypothesized. In many neurodegenerative disorders, ubiquitin-conjugated proteins increase in level and accumulate at the site of pathological lesions such as neurofibrillary tangles, amyloid plaques, and senile plaques. (Lennox et al., 1988; Lowe et al., 1988; for review, see Alves-Rodrigues et al., 1988). Accumulation of ubiquitinated proteins may result from energy deprivation inhibiting proteasome activity or, alternatively, a defect in the proteasome activity itself.

Alternatively, the toxic effect of β-amyloid could alter the control of protein degradation such that unregulated activity leads to early neuronal death. An example of such a possibility is any protein controlling apoptosis, such as Bcl-2. (Dimmeler et al., 1999). Proteasome-mediated degradation of Bcl-2 targeted by the dephosphorylation of the protein, which in turn signals the ubiquitination of Bcl-2, has been implicated in the events leading to apoptosis. (Dimmeler et al., 1999). On the other hand, apoptosis has been reported following exposure to, β-amyloid toxic fragments. (Yan et al., 1999). In addition, hyperactivity or overexpression of Bcl-2 leads to an increase of neuronal resistance to β-amyloid toxicity. (Saille al., 1999).

In spite of intensive research in this area, the critical molecular mechanisms underlying the neurological disorders known as the dementias are still far from understood, and means for preventing or ameliorating these devastating disorders have been elusive.

### SUMMARY OF THE INVENTION

The present invention demonstrates for the first time a link between β-amyloid neurotoxicity and proteasome-mediated protein degradation. It further shows that blocking either β-amyloid-mediated ubiquitination or proteasome activity effectively prevents the ability of β-amyloid to cause neuronal death. These results suggest a new approach for pharmacologic therapies for treatment of neurological diseases and disorders, in particular Alzbeimer's disease.

According to the invention, β-amyloid peptide causes the death of cortical neurons through the activation of protein degradation via the ubiquitin-proteasome proteolytic pathway, and those effects can be blocked. The invention involves a novel target for reducing β-amyloid peptide neuron toxicity, distinct from prior art relating to formation of β-amyloid peptide, in that it involves a different stage and mechanism of the overall process (from APP to β-amyloid peptide to Alzheimer's). Specifically, inhibitors used according to this invention are shown to act after β-amyloid peptide-formation. In contrast, the prior art relates only to inhibition occurring before β-amyloid peptide formation, presumably in the absence of β-amyloid peptide as disclosed in Fenteany et al. (U.S. Pat. No. 5,756,764). According to the present invention, a β-amyloid peptide-mediated ubiquitin-proteasome proteolysis inhibitor acts on a protein (or proteins) other than amyloid precursor protein in the presence of β-amyloid peptide.

The term “β-amyloid peptide” is intended to refer to a peptide fragment of 39 to 42 amino acids derived from any isoform of amyloid protein precursor, in particular to two principal forms of β-amyloid peptide, corresponding to the region 1-40 and 1-42 of the APP molecule, that are produced and accumulated in Alzheimer's brain. (Martin, 1999).

By “neurotoxic ubiquitin-proteasome proteolysis” is meant proteolysis that occurs through the ubiquitin-proteasome pathway that does occur normally, but is associated with a toxicity to neurons, particularly in a neurological disease or disorder. This term is inclusive of “β-amyloid peptide-mediated ubiquitin-proteasome proteolysis”, which is defined as proteolysis that occurs through the ubiquitin-proteasome pathway in the presence of β-amyloid peptide, and does not occur in its absence. According to the invention, β-amyloid peptide-mediated ubiquitin-proteasome proteolysis causes protein degradation that leads to early neuronal death and the consequent manifestations of Alzheimer's Disease.

Inhibiting ubiquitination and/or proteasome mediated proteolysis can prevent, alleviate, or block the progression of chronic neurodegeneration, in particular that due to β-amyloid peptide toxicity. The neurotoxic effect of β-amyloid peptide may be due to altering the control of protein-degradation such that unregulated activity leads to early neuronal death. The present invention provides that certain compounds may be able to block either proteasome activity (e.g., lactacystin) or ubiquitin activity (e.g., leucine-alanine dipeptide), thereby preventing β-amyloid peptide toxicity in a dosage dependent manner.

It is believed that the methods and compositions of the invention are widely applicable to many neurological diseases and disorders wherein β-amyloid peptide may not necessarily play a significant role. In many neurodegenerative disorders, ubiquitin-conjugated proteins are increased and accumulate at the site of pathological lesions. It is believed that administration of inhibitors of ubiquitin-proteasome proteolysis will also be effective to block the pathological effects resulting from such activity. Thus, the invention also includes a method for increasing neuronal viability and treating neurological diseases and disorders by administering an inhibitor of ubiquitin-proteasome proteolysis, wherein such proteolysis may be caused by factors other than the presence of β-amyloid peptide. Aspects of the invention that are described herein with respect to β-amyloid peptide-mediated ubiquitin-proteasome proteolysis should also be considered applicable more generally to neurodegenerative diseases or disorders wherein neurotoxic ubiquitin-proteasome proteolysis is present.

One aspect of the invention provides a method of blocking ubiquitination of neuronal proteins, particularly β-amyloid peptide-induced ubiquitination. β-amyloid peptide-induced ubiquitin to a target protein causes premature degradation of that protein. The ubiquitin-proteasome proteolytic pathway typically involves three enzymes operating successively to attach a peptide sequence know as ubiquitin to proteins that are destined for proteolytic processing. The ubiquitin sequence acts as a recognition sequence for the proteasome complex, and is associated with transport of the ubiquitinated protein to the proteolytic organelle known as the proteasome, which degrades the tagged protein or processes it accordingly. Leucine-alanine dipeptide is reportedly an inhibitor of ubiquitination that blocks the active site of the isopeptidase ligase and prevents it from binding ubiquitin to the target protein. (Reiss et al. (1988); Obin et al. (1999). Other inhibitors of ubiquitination have similar effects according to the invention. According to the invention, ubiquitination of intraneuronal protein is necessary but not sufficient to cause cell death, and must be coupled to proteasome-mediated protein-degradation to cause neurotoxicity. In Alzheimer's Disease, both steps are associated specifically with the presence of β-amyloid peptide.

In another aspect of the invention, β-amyloid peptide toxicity is blocked by administering a compound that inhibits proteasome activity. This feature is contrary to prior art suggestions that a dysfunctional proteasome causes increased ubiquitination in neurodegenerative disorders. According to the invention, the proteasome is functional and can be blocked, thereby reducing the formation of the end products causing β-amyloid peptide toxicity.

Lactacystin, an inhibitory compound, does not reduce ubiquitination of proteins. In fact, as demonstrated in the examples set forth hereinbelow, lactacystin may increase the level of ubiquitination by inhibiting proteasome activity, while blocking β-amyloid peptide toxicity.

Compounds that are suitable for blocking ubiquitination include leucine-alanine peptide and N-terminal analogs thereof. Compounds that are suitable for inhibiting proteasomal degradation of ubiquitinated protein include, for example, lactacystin, and analogs thereof. Lactacystin analogs include, for example, such compounds as defined in U.S. Pat. No. 5,756,764.

The terms “neurological disease or disorder” and “neurodegenerative disease or disorder” are intended to mean diseases associated with the brain and nervous system, including but not limited to, Alzheimer's disease, Parkinson's disease, Creutzfeld-Jacob Disease, Lewy Body Dementia, amyotrophic lateral sclerosis, stroke, epilepsy, multiple sclerosis, myasthenia gravis, Huntington's Disease, Down's Syndrome, nerve deafness, and Meniere's disease.). Other neurological diseases and disorders will be apparent to those of skill in the art and are encompassed by the definition as used in this invention. The invention is considered to be particularly applicable to the dementias, such as Alzheimer's Disease.

By “target neurons” is meant any population of neurons having neurotoxic ubiquitin-proteasome proteolysis, where it is desired to measure, reduce or eliminate such activity for the purposes of diagnosis, prevention and/or treatment, or research purposes. In a preferred embodiment such neurotoxic proteolysis is triggered by the presence of β-amyloid peptide. For treatment of Alzheimer's Disease and other dementias, such target neurons will generally be located in the brain, most particularly in the hippocampus and cortex.

According to the methods of the invention, the inhibitors of ubiquitination or proteasome activity may be administered alone or in combination, and may optionally be mixed with suitable carriers and excipients in pharmaceutical compositions, as will be evident to those of skill in the art. The compositions may be administered, for example, orally, parenterally and by inhalation, in the form of solutions or liquid suspensions, tablets or capsules, powders and the like. Suitable formulations and methods are known to those of skill in the pharmaceutical and medical arts.

It is one object of the invention to provide a method of suppressing the neurotoxic effect of β-amyloid peptide, comprising administering to a patient having β-amyloid peptide in a neurotoxic amount an inhibitor of β-amyloid peptide-mediated ubiquitin-proteasome proteolysis in an amount effective to suppress the neurotoxic effect of β-amyloid peptide. Preferably, the β-amyloid peptide-mediated ubiquitin-proteasome proteolysis acts on a target protein other than amyloid precursor protein. When administered in accordance with the invention, the inhibitor prevents β-amyloid peptide-induced morphologic degeneration of neurons, and does not affect neuronal viability in the absence of β-amyloid peptide.

In a preferred embodiment of this aspect of the invention, the inhibitor blocks β-amyloid peptide neurotoxicity in a concentration dependent manner.

Such inhibitors may be administered alone or in combination. In particularly preferred embodiments, the inhibitor is selected from the group consisting of lactacystin, lactacystin analogs, leucine-alanine dipeptide, leucine-alanine dipeptide analogs, and combinations thereof.

Preferably, the inhibitor has speific inhibitory effects on β-amyloid peptide-induced ubiquitin-proteasome proteolysis and reduces neurotoxicity, but does not affect other (non-neurotoxic) ubiquitin-proteasome proteolytic pathways.

According to the invention, the inhibitor may be used in vitro, for example, in isolated neural tissue or tissue culture, or may administered to a mammal, in particular a human.

The invention further includes a method of treating a neurodegenerative disease or disorder comprising administering to an individual in need of treatment an inhibitor of ubiquitin-proteasome proteolysis in an amount effective to suppress neurotoxic ubiquitin-proteosome proteolysis. In a preferred embodiment, the method comprises administering a sufficient amount of such inhibitor to block β-amyloid peptide-mediated ubiquitin-proteosome proteolysis, thereby blocking the neurotoxic effect of β-amyloid peptide. The method is particularly suitable for treating Alzheimer's disease.

The invention also includes a method of suppressing neurotoxicity comprising administering an inhibitor of ubiquitin-proteasome proteolytic activity in an amount effective to reduce ubiquitination and/or proteasomal activity. In one preferred embodiment, a sufficient amount of such inhibitor is administered to reduce or block β-amyloid peptide-induced ubiquitin-proteasome proteolytic activity. Preferred inhibitors for use in this aspect of the invention include lactacystin and analogs thereof, and leucine-alanine dipeptide and analogs thereof. According to one aspect of the invention, lactacystin inhibits β-amyloid peptide-induced proteasome activity thereby blocking β-amyloid peptide neurotoxicity, but does not reduce ubiquitination of proteins.

In contrast, leucine-alanine dipeptide inhibits β-amyloid peptide mediated ubiquitination. In particular, leucine-alanine dipeptide blocks the ubiquitin isopeptidase ligase, thereby preventing the attachment of ubiquitin to target proteins. In one preferred embodiment, leucine-alanine dipeptide is administered in a dose such that the concentration in the extracellular medium of the target cells is between 2 mM and 50 mM.

In a particularly preferred embodiment, lactacystin and leucine-alanine dipeptide (or their analogs) are administered in combination to inhibit both ubiquitination and proteasome activity.

The invention also includes a method of treating a neurodegenerative disease or disorder comprising administering an inhibitor of neurotoxic ubiquitin proteasome proteolytic activity in an amount effective to reduce ubiquitination and/or proteasomal activity to a patient in need of treatment, in particular a patient suffering from Atzheimer's disease. For treatment of Alzheitner's Disease, an amount sufficient to reduce or block β-amyloid peptide-induced ubiquitin-proteasome proteolytic activity is administered, thereby blocking the toxic effects of β-amyloid peptide on neurons.

Furthermore, the invention includes a method of treating or preventing a neurological disease or disorder comprising inhibiting neurotoxic ubiquitin-proteasome proteolysis, thereby reducing neuronal mortality. In a preferred embodiment, β-amyloid peptide-induced ubiquitin-proteasome proteolysis is reduced or blocked, to prevent or treat Alzheirner's Disease. According to this aspect of the invention, inhibiting β-amyloid peptide-induced proteolysis reduces neuronal protein degradation. This result is preferably achieved by administering an inhibitor compound, wherein the inhibition does not affect neuronal viability in the absence of β-amyloid peptide, but prevents β-amyloid peptide-induced morphologic degeneration of neurons, and blocks β-amyloid peptide toxicity in a concentration dependent manner. Such inhibition may be achieved by administering an inhibitor compound that blocks the toxic effect of β-amyloid peptide that stimulates protein-degradation. According to this aspect of the invention, such unregulated protein degradation leads to early neuronal death. The inhibitor compound may be an inhibitor that blocks β-amyloid peptide-mediated ubiquitination of bcl-2, thereby allowing bcl-2 to regulate and prevent apoptosis. By administering such an inhibitor, bcl-2 is prevented from being prematurely dephosphorylated, ubiquitinated, and degraded. Preferred inhibitors of this type are leucine-alanine dipeptide and N-terminal structural analogs thereof. Such inhibitors act by reducing β-amyloid peptide-induced ubiquitination of proteins.

The inhibitor compound may also be an inhibitor of post-ubiquitination proteasome activity. Preferred inhibitors of this type are lactacystin and analogs thereof. The effective amounts of lactacystin or an analog thereof for particular individuals and medical conditions can be determined by routine experimentation by persons of skill in the medical and pharmaceutical arts. In one preferred embodiment, lactacystin is administered to a patient in an amount such that the extracellular concentration for the target neuronal cells is between 1 nM to 500 nM. More preferably, the concentration around the cells is between 25 nM and 500 nM. Most preferably, the concentration will be between 100 nM and 500 nM. Ideally, an amount will be administered such that toxicity is reduced to β-amyloid peptide-free control levels.

Preferably, the inhibitor of post-ubiquitination proteasome activity, when used alone, does not reduce ubiquitination of proteins, but rather, inhibits proteasome activity downstream from ubiquitination.

The invention also includes a method for preventing or treating a neurological disease or disorder comprising inhibiting neurotoxic ubiquitin-proteasome proteolysis, particularly β-amyloid peptide-induced ubiquitin-proteasome proteolysis, by administering an inhibitor of ubiquitination together with an inhibitor of post-ubiquitination proteasome activity. In a preferred embodiment, the method comprises administering lactacystin and leucine-alanine dipeptide together.

The invention also includes a pharmaceutical composition comprising a neuroprotective combination of an inhibitor of ubiquitination and an inhibitor of post-ubiquitination proteasome-mediated proteolysis. In a preferred embodiment, the composition includes lactacystin and leucine-alanine dipeptide together, optionally with pharmaceutically acceptable excipient(s) and/or carrier(s), in an effective dosage amount to inhibit β-amyloid peptide-induced ubiquitin-proteasome proteolysis in the target neurons.

The invention also includes a method of reducing the neurotoxic effect of β-amyloid peptide and increasing the viability of neurons containing toxic concentrations of β-amyloid peptide, comprising reducing proteasome-mediated proteolysis. The method comprises administering an effective amount of an inhibitor of ubiquitination and/or an inhibitor of post-ubiquitination proteolysis to reduce the neurotoxic effects and increase neuronal viability. Fluorescein diacetate uptake should be increased and propidium staining reduced in effectively treated neurons.

### BRIEF DESCRIPTION OF THE DRAWINGS

The invention is better understood by reading the following detailed description with reference to the accompanying figures, in which like references refer to like elements throughout, and in which:

FIGS. 1A, 1B, 1C and 1D provide morphologic analysis of β-amyloid-induced cortical neuron damage shown in representative phase-contrast images of cortical neurons at 13 days in vitro. Cells are treated for 5 days with vehicle (FIG. 1A), 50 nM lactacystin (FIG. 1B), 20 μM β-amyloid (FIG. 1C), or 20 μM β-amyloid+50 nM lactacystin (FIG. 1D).

FIGS. 2A, 2B, 2C, 2D, 2E, 2F, 2G, 2H, 2I, 2L, 2M and 2N demonstrate viability of cortical neurons. β-Amyloid (β-am) treatment significantly reduces the number of viable cells. Cells are treated with the agents indicated in the respective heading (e.g. LACTA for lactacystin) and then stained with fluorescein and propidium. FIGS. 2A, 2D, 2G and 2L represent fields of cells observed in epifluorescence with a fluorescein filter. FIGS. 2B, 2E, 2H, and 2M show propidium labeling in the same microscopic field obtained with a rhodamine filter set. FIGS. 2C, 2F, 2I and 2N provide quantitative analyses of data obtained from cell counts. Data are mean±SE (bars) values. In total, 600 cells are counted for each treatment (f=1,488.43, * p<0.05 versus control cells; f=1,021.44, ▪ p<0.05 versus β-am alone by ANOVA and t test.

FIGS. 3A and 3B show inhibition of β-amyloid (β-am) neurotoxicity by lactacystin (lacta) and Leu-Ala. Cell viability is assessed using the MTT assay. The conversion of tetrazolium salts is measured using a spectrophotometer and expressed as absorbance in arbitrary units. FIG. 3A shows a pharmacological profile of the protective effect of lacta. Lacta inhibits the neurotoxic effect cause by β-am in a concentration-dependent manner (•&Rectsolid;•). FIG. 2B shows the effect of Leu-Ala and lacta on β-am toxicity. β-am treatment greatly decreases viable cells (second column), whereas 2 mM Leu-Ala (third column) and 50 nM lacta (fourth column) cause a clear reduction of the cell damage caused by β-am. * p<0.05 versus control cells, ▪ p<0.01 versus β-am alone; f=2.02, 2.015, 2.015, 2.015, and 2.036 for 10, 25, 50, 100, and 500 nM lacta, respectively.

FIG. 4 demonstrates the effect of β-amyloid on ubiquitination of neuronal proteins. Control cells (lane 1) shows the absence of ubiquitinated protein, whereas β-amyloid-treated cells shows a dramatic increase in level of ubiquitinated proteins ranging in size from 14 to 50 kDa (lane 2). Lactacystin alone (lane 3), by blocking proteasome activity, causes an accumulation of ubiquitinated protein, although to a lesser extent than β-amyloid. In addition, as expected, lactacystin does not affect ubiquitination induced by β-amyloid (lane 3). Leu-Ala alone does not affect protein ubiquitination in control cells (lane 5). However, β-amyloid-induced ubiquitination is markedly reduced by Leu-Ala (lane 6).

### DETAILED DESCRIPTION OF THE INVENTION

In describing preferred embodiments of the present invention, specific terminology is employed for the sake of clarity. However, the invention is not intended to be limited to the specific terminology so selected. It is to be understood that each specific element includes all technical equivalents, which operate in a similar manner to accomplish a similar purpose. Each reference cited here is incorporated by reference as if each were individually incorporated by reference.

Experiments conducted in relation to this invention demonstrate that β-amyloid peptide acts to induce aberrant ubiquitination and/or proteasomal formation, which leads to protein degradation, and therefore neuronal death. This process, which may be referred to as neurotoxic ubiquitin-proteasome proteolysis, may be blocked by either inhibiting the ubiquitination step (by utilizing leucine-alanine dipeptide), or by inhibiting proteasome activity (e.g., by utilizing lactacystin), or both.

These experiments show that lactacystin and leucine-alanine dipeptide have inhibitory effect on β-amyloid peptide-induced neurotoxicity. Such compounds have clinical value in individuals suffering from neurodegenerative diseases and disorders, such as Alzheimer's Disease. The present invention also discloses a method of preventing β-amyloid peptide-induced neurotoxicity by blocking ubiquitin-proteasome proteolysis.

According to the invention, inhibitory compounds prevent β-amyloid peptide-induced morphologic degeneration of neurons, but do not affect neuronal viability in the absence of β-amyloid peptide. In addition, the inhibitory compounds block β-amyloid peptide toxicity in a concentration dependent manner.

The present invention provides a method of treating or preventing neurological diseases by reducing β-amyloid peptide-induced neuronal mortality by suppressing the ubiquitin-proteasome proteolytic pathway. This suppression can be achieved by administering an inhibitory compound and preventing neuronal protein degradation. These compounds do not affect neuronal viability in the absence of β-amyloid peptide, but prevent β-amyloid peptide-induced morphologic degeneration of neurons, and block βAP toxicity in a concentration-dependent manner.

In one embodiment of the invention, 2 mM leucine-alanine dipeptide, or an N-terminal structural analog, is used to reduce βAP induced ubiquitination of proteins. Because leucine-alanine dipeptide inhibits ubiquitination, it is referred to as an inhibitor of pre-ubiquitination proteasome activity. On the other hand, lactacystin is an inhibitor of post-ubiquitination proteasome activity. If used alone, lactacystin does not reduce ubiquitination of proteins, but inhibits proteasome activity downstrean from ubiquitination. Preferably an amount of lactacystin or an analog thereof is administered that is sufficient to produce a concentration of 1 nM to 500 nM in the extracellular medium of the target neurons, more preferably a concentration of 25 nM to 500 nM. When administered in sufficient concentration, lactacystin reduces toxicity to β-amyloid peptide-free control levels.

In addition, an inhibitor of ubiquitination can be administered together with an inhibitor of post-ubiquitination proteasome activity, such as administering lactacystin and leucine-alanine dipeptide together, to produce inhibition of β-amyloid peptide-induced ubiquitin-proteasome proteolysis. This may be used to treat or prevent neurodegenerative disorders, such as Alzheimer's disease. Moreover, an inhibitor of ubiquitination and an inhibitor of post-ubiquitination proteasome-mediated proteolysis may be combined in a pharmaceutical composition to be used in this manner as a neuroprotective composition.

The present invention further provides a method of diagnosing a neurodegenerative disease or disorder comprising determining the level of β-amyloid peptide-mediated proteasome activity, and a method of increasing the viability of neurons containing toxic concentrations of β-amyloid peptide, comprising reducing proteasome-mediated proteolysis. Moreover, the invention provides a method of reducing the neurotoxic effect of β-amyloid peptide comprising reducing proteasome-mediated proteolysis, wherein reduced toxicity may be demonstrated, inter alia, by increased fluorescein diacetate uptake and/or reduced propidium staining of neural tissue.

The invention also provides a method of reducing toxicity and/or increasing neuronal survival comprising reducing or blocking neurotoxic ubiquitin-proteasome proteolysis by administering an effective dose of a ubiquitination inhibitor or a proteasome inhibitor. This method can be used for the prevention and/or treatment of neurodegenerative diseases and disorders, in particular Alzheimer's Disease.

The embodiments illustrated and discussed in the present specification are intended only to teach those skilled in the art the best way known to the inventors to make and use the invention, and should not be considered as limiting the scope of the present invention. The exemplified embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

### EXAMPLES

Materials

All the substances were obtained from Sigma unless otherwise specified. β-Amyloid 1-40 was obtained from Bachem (Switzerland) and prepared as suggested from the manufacturer. In brief, the peptide was dissolved in water at a 1 mM concentration; 3 days before the experiment it was diluted with PBS at 500 μM and kept at 37° C. until added to the cultures. Anti-ubiquitin antibody was purchased from Sigma Chemical Co. (St. Louis, Mo., USA, Catalogue no. U-5379).

Preparation of Rat Cortical Neurons

Neurons were prepared from 17-day-old rat fetuses, according to a previously published protocol (Hampson et al. (1998)). In brief, the fetuses were decapitated, the brains were dissected and placed in phosphate-buffered saline (PBS) containing 4.5 g/L glucose. Hemispheres were separated, and meninges were carefully removed. Cortical tissues were freed from subcortical structures and cut into small fragments. Tissues were incubated with papain activated with cysteine for 10 min at 37° C. Papain was neutralized with a solution of ovomucoid and bovine serum albumin. Finally, tissues were mechanically dissociated until a single-cell suspension was obtained. Cells were plated in poly-D-lysine-coated 3.5-cm-diameter Petri dishes, 48-multiwell plates, or tissue culture flasks.

Viability Assessment

Viability of cortical neurons was assessed with a propidium iodide exclusion test and fluorescein diacetate incorporation assessment, according to a previously published protocol. (Favit et al. (1992)). Rat cortical neurons seeded on 3.5-cm-diameter dishes were treated, washed with PBS, and incubated with a PBS solution containing 36 μM fluroescein diacetate and 7 μM propidium iodide for 3 min at room temperature. Cell viability was assessed by counting 100 cells per microscope field (using a 20× lens) for the number of fluorescein-labeled neurons versus propidium-positive cells. Experiments were conducted in duplicate and repeated at least three times. Statistical validity was assessed by ANOVA followed by a post hoc test.

3-(4,5-Dimethylthiazol-2-yl)-2,5-diphenyltetrazolium bromide (MTT) Neurotoxicity Assay

Neurons plated at a density of 0.4×10⁶ in 48-multiwell plates were exposed for the appropriate times to the test agents. MTT reduction was used to measure mitochondrial activity as an index of cell viability. The reaction was started by adding 0.4 mg/ml MTT in a PBS solution to the neurons. After 16 h of incubation at 37° C., 100 μl of pure dimethyl sulfoxide is added to each well. After an additional 12 h of incubation, absorbance values at 570 nm were determined with an automatic microplate reader, using 630 nm as a reference wavelength. Experiments were done in 48-multiwell plates allowing eight samples for each experimental point. Experiments were repeated at least three times. Statistical validation was assessed by ANOVA followed by a post hoc test.

Western Blots

Western blot analysis was performed as previously described (Favit et al., 1998) with the following modifications. Proteins were extracted from neuronal pellets in a PBS solution containing 1 mg/ml leupeptin, 1 mg/ml pepstatin, 1 mg/ml chymostatin, 1 mg/ml antipain, and 0.2 mM phenylmethylsulfonyl fluoride. (Vannucci et al. (1998)). The crude homogenate was balanced with sample buffer containing 0.5 M Tris-HCl (pH 6.8), 10% glycerol, 2% sodium dodecyl sulfate, and 0.5% 2-mercaptoethanol to a final volume of 20 μl with a total protein concentration of 10 μg/hl. Sodium dodecyl sulfate-polyacrylamide gel electrophoresis was carried out in a 4-20% acrylamide gradient gel 1.5 mm thick. (Novex, San Diego, Calif., U.S.A.). Twenty micrograms per lane of protein of the crude homogenate was electrophoresed and transferred overnight onto a nitrocellulose membrane (Schleicher & Schuell). The membrane was blocked in 1% bovine serum albumin in 95% Tris-buffered saline for 1 h. Immunodetection of ubiquitin was obtained by exposing the membrane to an anti-ubaiqtin polyclonal primary antibody at 1:1,000 dilution for 1 h. Blots were then incubated with an anti-mouse alkaline phosphatase-conjugated secondary antibody for 1 h. Finally, the nitrocellulose was stained with a solution containing 0.1 M Tris-HCl (pH 9.6), 0.001 M MgCl₂, 1% nitro blue terrazolium (Pierce), and 1% 5-bromo-4chloro-3-indolyl phosphate toluidine salt (Pierce). All reactions were carried out at room temperature. At least three independent sets of experiments were conducted.

Results

Prevention of β-Amyloid-Induced Neuronal Degeneration by Antiproteasome Agents

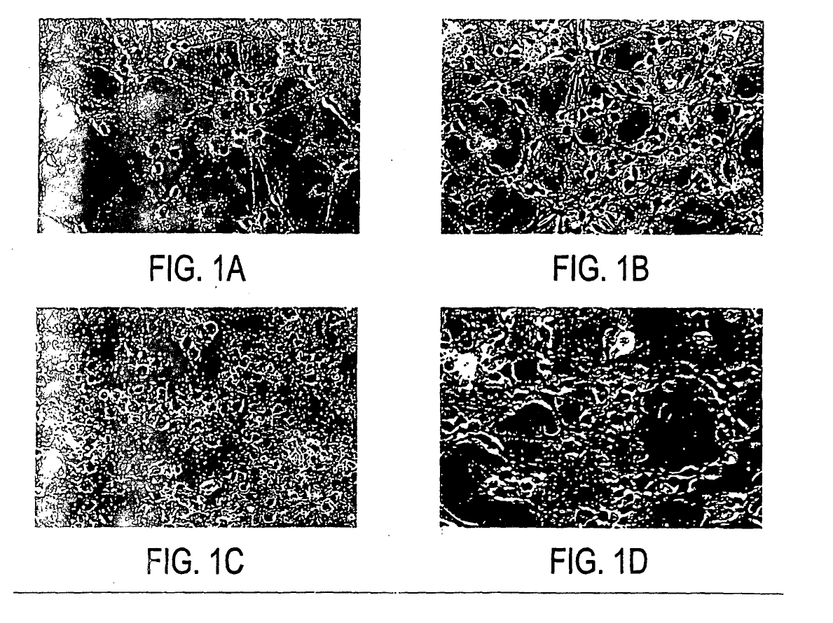

Exposure to the activated β-amyloid fragment 1-40 (β-amyloid) at 20 μM for 5 days causes degeneration of cortical neurons as shown in FIG. 1C. Parallel exposure to either the scrambled or the reversed peptide did not cause any significant degenerative changes. The presence of either 50 nM lactacystin (Biomol), an inhibitor of ubiquitination, prevented β-amyloid-induced morphologic degeneration of neurons. Lactacystin (FIG. 1B) or Leu-Ala alone did not affect neuronal viability.

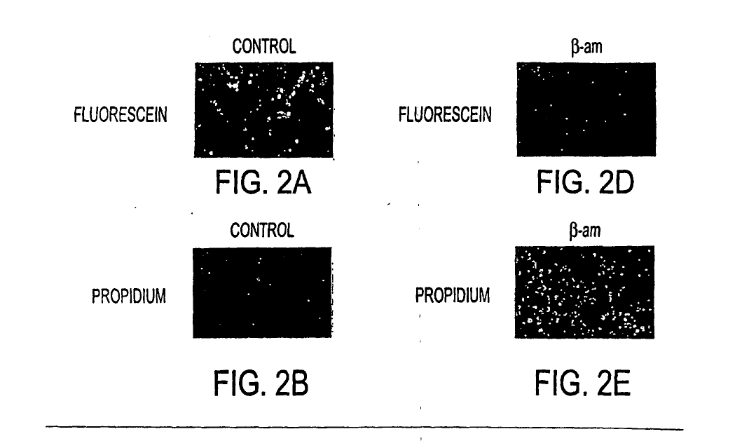

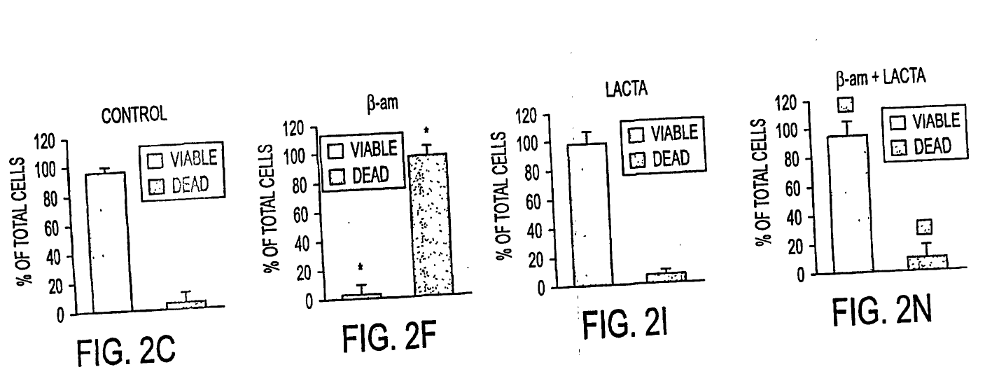

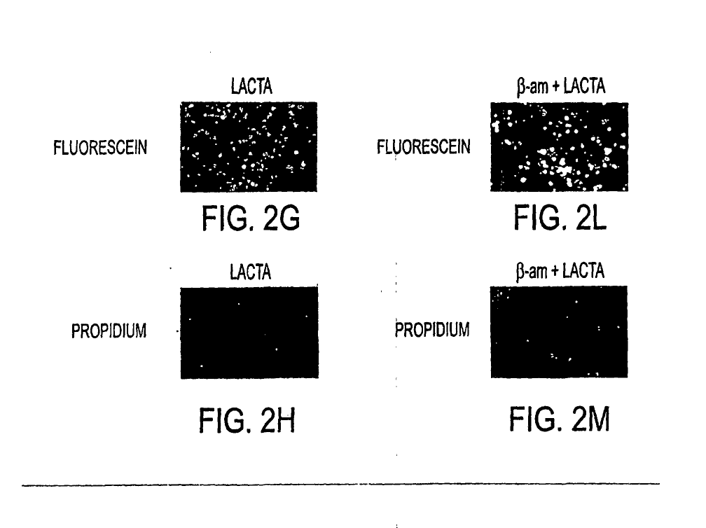

Viability of cortical neurons was assessed by testing the ability of living cells to take up the vital dye fluorescein diacetate and to exclude propidiurn iodide. Cells treated with β-amyloid are not able to sequester fluorescein (FIG. 2D), an effect consistent with significant cell degeneration. Neurons exposed to β-amyloid in the presence of 50 nM lactacystin, however, show fluorescein uptake similar to that of control cells (FIG. 2G). Propidiurn labeling is used as an indicator of cell damage. Neurons treated with β-amyloid show clear staining with propidium iodide (FIG. 2E), consistent with a high degree of damage, including cellular and nuclear membrane permeabilization. In addition, when combined with 50 nM lactacystin, β-amyloid-induced propidium staining is reversed to near control levels (FIG. 2M). The neurotoxic effects of β-amyloid and their prevention by lactacystin as assessed by fluorescein and propidium staining are also quantified and statistically validated by comparing the number of fluorescein—versus propidium-stained cells (FIGS. 2C, 2F, 2I and 2N).

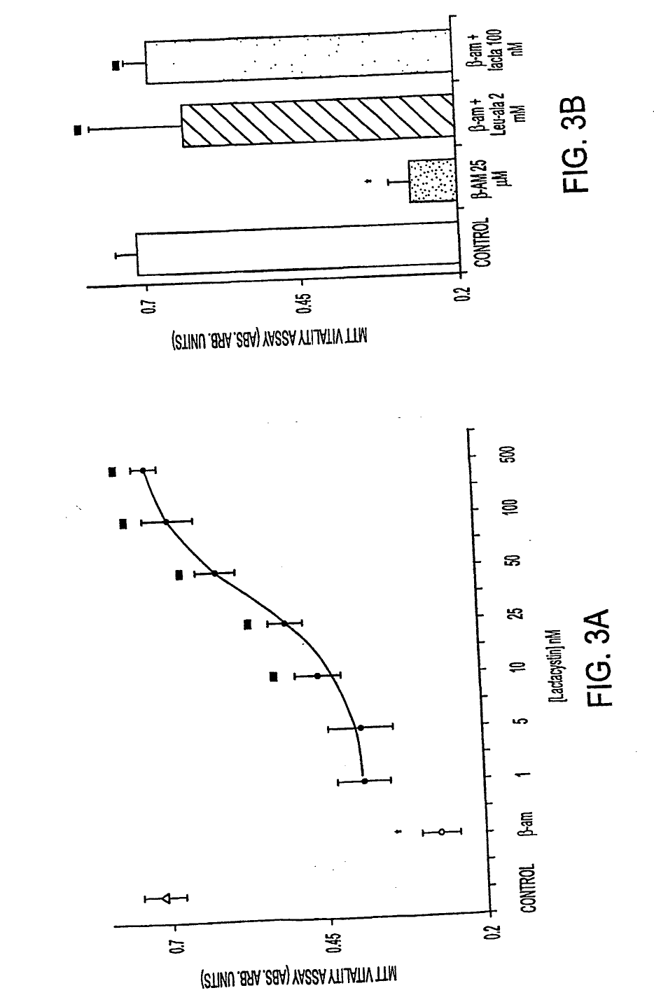

β-Amyloid toxicity is blocked by lactacystin in a concentration-dependent manner (FIG. 3A). The effect of lactacystin is already statistically significant at 25 nM and reaches an apparent plateau at 500 nM. The apparent EC₅₀ of the drug was 30 nM. At the highest concentration tested, the compound almost entirely prevents the toxic effect of β-amyloid (FIG. 3A). A comparison between the effect of lactacystin and Leu-Ala is displayed in FIG. 3B. In addition, the reverse dipeptide Ala-Leu does not cause any reduction of β-amyloid toxicity (data now shown).

Effect of β-Amyloid on Ubiquitination of Intracellular Proteins

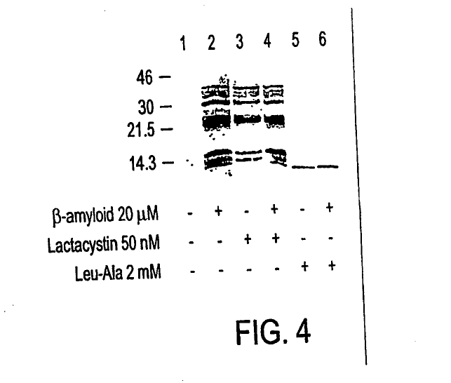

Blocking ubiquitination with Leu-Ala or inhibiting proteasome activity with lactacystin prevent β-amyloid-induced neurotoxicity. This suggests that β-amyloid-triggered toxicity is mediated by activation of proteasome-mediated protein degradation. Therefore, the ability of β-amyloid to cause ubiquitination of neuronal proteins was tested. Neurons were incubated with β-amyloid for 72 h in the presence of 50 nM lactacystin or 2 mM Leu-Ala and harvested. Western blot analysis shows that the untreated cells have very few ubiquitinated proteins (FIG. 4, lane 1). β-Amyloid-treated neurons, however, show a marked increase of ubiquitination of multiple proteins having molecular weights between about 14 kD and about 50 kD (FIG. 4, lane 2). Blocking proteasonie activity with lactacystin treatment also increases the presence of ubiquitinated protein as a result of the accumulation of basically ubiquitinated protein (FIG. 4, lane 3). β-Amyloid-induced ubiquitination is not affected by lactacystin, which blocks the activity of the proteasome downstream of ubiquitination. β-amyloid- induced ubiquitination, however, is completely prevented by treating the cultures with Leu-Ala, which directly blocks the ubiquitin isopeptidase ligase, thereby preventing the attachment of ubiquitin to target proteins (Obin et al., 1999) (FIG. 4, lane 6). As a consequence of this, the target proteins are not degraded by the proteasome.

Discussion

Experiments conducted in relation to the present invention show that β-amyloid toxicity can be prevented by agents that block either ubiquitination or proteasome activity. Furthermore, these experimental data support the hypothesis that ubiquitination of intraneuronal proteins is not sufficient to cause cell death but must be coupled to proteasome-mediated protein degradation because lactacystin and β-amyloid both cause a similar pattern of protein ubiquitination. Yet, lactacystin does not cause toxicity. The increase of ubiquitinated proteins in the cells may induce a compensatory negative feedback to reduce further ubiquitination, if ubiquitinated proteins are not efficiently removed. Therefore, ubiquitination in the presence of both β-amyloid and lactaystin does not increase above β-amyloid or lactacystin alone treatment.

It is believed that these findings can be extended generally to include neurotoxic ubiquitin-proteasome proteolysis occurring in a number of neurodegenerative diseases and disorders, particularly those in which amyloid proteins are involved. Pharmacological inhibition of protein ubiquitination and degradation should prevent, alleviate, or even block

the progression of chronic neurodegeneration associated with long-term neurologic pathologies such as Alzheimer's disease.

References cited herein are listed below for convenience and are hereby incorporated by reference.

### REFERENCES

Alves-Rodrigues A., Gregori L., and Figueiredo-Pereira M. E. (1998) Ubiquitin, cellular inclusions and their role in neurodegeneration. Trends Neurosci. 21, 516-520.

Ciechanover A. and Schwartz A. L. (1994) The ubiquitin-mediated proteolytic pathway: mechanisms of recognition of the proteolytic substrate and involvement in the degradation of native cellular proteins. FASEB J. 8, 182-191.

Ciechanover A. (1994) The ubiquitin-proteosome proteolytic pathway. Cell 79, 13-21.

Davis J. B. (1996) Oxidative mechanisms in beta-amyloid cytotoxicity. Neurodegeneration 5, 441-444; Suzuki A. (1997) Amyloid β-protein induces necrotic cell death mediated by ICE cascade in PC12 cells. Exp. Cell Res. 234, 507-511.

Dimmeler S., Breitschopf K., Haendeler J., and Zeiher A. M. (1999) Dephosphorylation targets Bcl-2 for ubiquitin-dependent degradation: a link between the apoptosome and the proteasome pathway. J. Exp. Med. 11, 1815-1822.

Favit A., Grimaldi M., Nelson T. J., and Alkon D. L. (1998) Alzheimer's-specific effects of soluble β-amyloid on PKCα and PKCγ degradation in human fibroblasts. Proc. Natl. Acad. Sci. USA 95, 5562-5567.

Favit, A., Grimaldi, M., and Alkon, D. L. (2000) Prevention of beta-amyloid neurotoxicity by blockade of the ubiquitin-proteasome proteolytic pathway, J Neurochem. 75(3), 1258-1263.

Favit A., Nicoletti F., Scapagnini U., and Canonico P. L. (1992) Ubiquinone protects cultured neurons against spontaneous and excitotoxin-induced degeneration. J. Cereb. Blood Flow Metab. 12, 638-645.

Fenteany G., Standaert R. F., Lane W. S., Choi S., Corey E. J., and Schreiber S. L. (1995) Inhibition of proteosome activities and subunit-specific amino-terminal threonine modification by lactacystin. Science 268, 726-731.

Hampson A. J., Grimaldi M., Axelrod J., and Wink D. (1998) Cannabidiol and (--) Delta-9-tetrahydrocannabinol are neuroprotective antioxidants. Proc. Natl. Acad. Sci. USA 95, 8268-8273.

Honda T, Yasutake K, Nihonmatsu N, Mercken M, Takahashi H, Murayama O, Murayama M, Sato K, Omori A, Tsubuki S, Saido TC, Takashima A. (1999) Dual roles of proteasome in the metabolism of presenilin 1. J. Neurochem 72(1):255-61.

Laney J. D. and Hochstrasser M. (1999) Substrate targeting in the ubiquitin system. Cell 97, 427-430.

Lennox G., Lowe J., Morrel K., Landon M., and Mayer R. J. (1988) Ubiquitin is a component of neurofibrillary tangles in a variety of neurodegenerative disease. Neurosci. Lett. 94,211-217.

Lowe J., Blanchard A., Morrell K., Lennox G., Reynolds L., Billett M., Landon M., and Mayer R. J. (1988) Ubiquitin is a common factor in intermediate filament inclusion bodies of diverse type in man, including those of Parkinson's disease, Pick's disease, and Alzlieimer's disease, as well as Rosenthal fibers in cerebellar astrocytomas, cytoplasmic bodies in muscle, and Mallory bodies in alcoholic liver disease. J. Pathol. 155, 9-15.

Marambaud, P., Lopez-Perez, E., Wilk, S., Checler, F. (1997) Constitutive and protein kdnase C-regulated secretory cleavage of Alzheimer's β-amyloid precursor protein: different control of early and late events by proteasome. Journal of Neurochemisthy 69:6, 2500-2505.

Martin J. B. (1999) Molecular basis of the neurodegenerative disorders. N. Engl. J. Med. 340, 1970-1980.

Mattson M. P. and Pedersen W. A. (1998) Effects of amyloid precursor protein derivatives and oxidative stress on basal forebrain cholinergic systems in Alzheimer's disease. Ira. J. Dev. Neurosci. 16, 737-753.

Obin M., Mesco E., Gong X., Hass A. L., Joseph., and Taylor A. (1999) Neurite outgrowth in PC12 cells: distinguish the roles of ubiquitinylation and ubiquitin-dependent proteolysis. J. Biol. Chern. 274, 11789-11795.

Reiss Y., Kaim D., and Hershko A. (1988) Specificity of binding of NH₂-terminal residue of proteins to ubiquitin-protein ligase. Use of amino acid derivatives to characterize specific binding sites. J. Biol. Chem. 263, 2693-2698.

Saille C., Marin P., Martinou J. C., Nicole A., London J., and Ceballos-Picot I. (1999) Transgenic murine cortical neurons expressing human Bcl-2 exhibit increased resistance to amyloid beta-peptide neurotoxicity. Neuroscience 92, 1455-1463.

Vannucci S. J., Mummery R, Hawkes R. B., Rider C. C., and Beesley P. W. (1998) Hypoxia-ischemia induces a rapid elevation of ubiquitin conjugate levels and ubiquitin immtuoreactivity in the immature rat brain. J. Ceereb. Blood Flow Metab. 18, 376-385.

Vickers J. C., Dickson T. C., Adlard P. A., Saunders H. L., King C. E., and McCormack G. (2000) The cause of neuronal degeneration in Alzheimer's disease. Prog. Neurobiol. 60, 139-165.

Wilson C. A., Doms R. W., and Lee V. M. (1999) Intracellular APP processing and A beta production in Alzheimer's disease. J. Neuropathol. Exp. Neurol. 58, 787-794.

Yamazaki T, Haass C, Saido T C, Omura S, Ihara Y. (1997) Specific increase in amyloid beta-protein 42 secretion ratio by calpain inhibition. Biochemistry 36(27):8377-83.

Yan X. Z., Qiao J. T., Dou Y., and Qiao Z. D. (1999) β-Amyloid peptide fragment 31-35 induces apoptosis in cultured cortical neurons. Neuroscience 92, 177-184.

We claim:

1. A method of reducing neurotoxic effects of β-amyloid peptide, comprising administering to neuronal cells containing neurotoxic levels of β-amyloid peptide an inhibitor of ubiquitination in an amount effective to reduce the neurotoxic effect of β-amyloid peptide.

2. The method of claim 1 wherein the inhibitor is leucine-alanine dipeptide.

3. The method of claim 1, wherein said inhibitor suppresses the formation of ubiquitinated neuronal proteins.

4. The method of claim 3, wherein said proteins have molecular weights between about 14 kD and about 50 kD.

5. The method of claim 1, that additionally comprises administering to the neuronal cells an inhibitor of post-ubiquitination proteasomal activity in an amount sufficient to inhibit proteasomal processing of ubiquitinated protein.

6. The method of claim 5 wherein the inhibitor of ubiquitination is leucine-alanine dipeptide and the inhibitor of post-ubiquitination proteasomal activity is lactacystin.

7. A pharmaceutical composition comprising an inhibiting amount of an inhibitor of protein ubiquitination and an inhibitor of post-ubiquitination proteasome-mediated proteolysis.

8. The pharmaceutical composition of claim 7, wherein the inhibitor of ubiquitination is leucine-alanine dipeptide or an analog thereof.

9. The pharmaceutical composition of claim 7, wherein the inhibitor of post-ubiquitination proteasome-mediated proteolysis is lactacystin or an analog thereof.

10. The pharmaceutical composition of claim 9, wherein the inhibitor of ubiquitination is leucine-alanine dipeptide.

11. A method of reducing the neurotoxic effect of β-amyloid peptide, comprising administering to neuronal cells containing neurotoxic levels of β-amyloid peptide an inhibitor of β-amyloid peptide-mediated ubiquitin-proteasome proteolysis in an amount effective to reduce the neurotoxic effect of β-amyloid peptide.

12. The method of claim 11, wherein the β-amyloid peptide-mediated ubiquitin-proteasome proteolysis acts on a target protein other than amyloid precursor protein.

13. The method of claim 11, wherein the inhibitor prevents β-amyloid peptide-induced morphologic degeneration of neurons.

14. The method of claim 11, wherein the inhibitor does not affect neuronal viability in the absence of β-amyloid peptide.

15. The method of claim 11, wherein the inhibitor blocks β-amyloid peptide neurotoxicity in a concentration dependent manner.

16. The method of claim 11, wherein the inhibitor is selected from the group consisting of lactacystin, a lactacystin analog, leucine-alanine dipeptide, a leucine-alanine dipeptide analog, and combinations thereof.

17. The method of claim 11, wherein the inhibitor is administered to a mammal.

18. The method of claim 17, wherein the mammal is a human.

19. A method of inhibiting neurotoxic ubiquitin-proteasome proteolysis comprising administering to neuronal cells an inhibitor of ubiquitination in an effective amount.

20. The method of claim 19 wherein the inhibitor is leucine-alanine dipeptide.

21. The method of claim 19, wherein said inhibitor suppresses the formation of ubiquitinated neuronal proteins.

22. The method of claim 21, wherein said proteins have molecular weights between about 14 kD and about 50 kD.

23. The method of claim 19, that additionally comprises administering to the neuronal cells an inhibitor of post-ubiquitination proteasomal activity in an amount sufficient to inhibit proteasomal processing of ubiquitinated protein.

24. The method of claim 23 wherein the inhibitor of ubiquitination is leucine-alanine dipeptide and the inhibitor of post-ubiquitination proteasomal activity is lactacystin.

25. The method of claim 19, wherein the inhibitor is administered to a mammal.

26. The method of claim 19, wherein the mammal is a human.

27. The method of claim 19, wherein said neurotoxic ubiquitin-proteasome proteolysis is β-amyloid peptide-mediated ubiquitin-proteasome proteolysis.

28. A method of treating a neurodegenerative disease or disorder comprising administering to an individual in need of treatment an inhibitor of neurotoxic peptide-mediated ubiquitin-proteasome proteolysis in an effective amount.

29. The method of claim 28, wherein the neurotoxic peptide-mediated ubiquitin-proteasome proteolysis is β-amnyloid peptide-induced ubiquitin-proteasome activity.

30. The method of claim 29, wherein the inhibitor is an inhibitor of ubiquitination.

31. The method of claim 29, wherein the neurodegenerative disease is Alzheimer's disease.

32. The method of claim 31, that additionally comprises administering an inhibitor of post-ubiquitination proteolysis.

33. A method of suppressing neurotoxicity comprising administering to neuronal cells challenged with neurotoxic levels of β-amyloid peptide an inhibitor of β-amyloid peptide-induced ubiquitin-proteasome proteolytic activity in an amount effective to reduce ubiquitination and/or proteasomal activity.

34. The method of claim 33, wherein the inhibitor is lactacystin.

35. The method of claim 34, wherein lactacystin is administered in an amount such that the extracellular concentration for target neuronal cells is between 1 nM and 500 nM.

36. The method of claim 35, wherein lactacystin is administered in an amount such that the extracellular concentration for target neuronal cells is between 25 nM and 500 nM.

37. The method of claim 34, wherein lactacystin inhibits β-amyloid peptide-inducement of proteasome activity thereby blocking β-amyloid peptide neurotoxicity.

38. The method of claim 33, wherein the inhibitor is leucine-alanine dipeptide.

39. The method of claim 38, wherein leucine-alanine dipeptide inhibits β-amyloid peptide mediated ubiquitination.

40. The method of claim 39, wherein leucine-alanine dipeptide blocks the ubiquitin isopeptidase ligase, thereby preventing the attachment of ubiquitin to target proteins.

41. The method of claim 33, wherein lactacystin and leucine-alanine dipeptide are administered in combination to inhibit both ubiquitination and proteasome formation.

42. The method of claim 33 wherein cell mortality is reduced.

43. A method of increasing the viability of neurons containing toxic concentrations of β-amyloid peptide, comprising administering an effective amount of an inhibitor of β-amyloid peptide-mediated ubiquitin-proteasome proteolysis such that the viability of the neurons is increased.

44. The method of claim 43, wherein fluorescein diacetate uptake of the neurons is increased.

45. The method of claim 43, wherein propidium staining of the cells is reduced.
